(19) United States
(12) Patent Application Publication
(10) Pub. No.: US 2025/0269355 A1
(43) Pub. Date: Aug. 28, 2025

(54) Method and device for preparing alumina support with reducing function and catalyst using carbon containing mixed gas

(71) Applicants: Quzhou Resource and Chemical Innovation Research Institute, Quzhou City (CN); Beijing University of Chemical Technology, Beijing (CN)

(72) Inventors: Junting Feng, Beijing (CN); Xue Duan, Beijing (CN); Yijun Zhang, Beijing (CN); Qian Wang, Beijing (CN); Yuanyuan Gao, Beijing (CN); Dianqing Li, Beijing (CN)

(21) Appl. No.: 19/063,645

(22) Filed: Feb. 26, 2025

(30) Foreign Application Priority Data
Feb. 27, 2024 (CN) 202410216938.0

(51) Int. Cl. B01J 21/04; B01J 37/02; B01J 37/08; B01J 37/16

(52) U.S. Cl. CPC B01J 21/04; B01J 37/0201; B01J 37/08; B01J 37/16

(57) ABSTRACT

The invention provides a method and device for preparing alumina support with reducing function and catalyst using carbon containing mixed gas. The preparation steps of alumina support with reducing function and catalyst are as follows: (1) Place the alumina precursor in a reactor and react it with a carbon containing mixed gas at 400-600° C. Control the gas flow rate and reaction time to obtain alumina support with reducing function. (2) Metal is loaded by impregnation method, and the metal is reduced in situ during the loading process to obtain precious metal catalysts. At the same time, a device for preparing alumina support with reducing function using carbon containing mixed gas is provided. The prepared alumina support with reducing function can accurately control the in-situ reduction of precious metals on the support during the impregnation process, avoiding the traditional unit operation process of high energy consumption and dangerous first loading and then hydrogenation reduction.

### CROSS REFERENCE TO RELATED APPLICATION

This application claims priority benefit of Chinese patent Application No. 202410216938.0, filed on Feb. 27, 2024, and the entire contents of which are incorporated herein by reference.

### TECHNICAL FIELD

The present invention relates to the field of catalyst preparation, specifically to the preparation of an alumina support with reducing function and a simple preparation technology for precious metal catalysts.

### BACKGROUND ART

Catalysts are the “heart” and foundation of catalytic reactions. Compared to non-precious metal catalysts, precious metal catalysts are highly valued for their excellent activity, selectivity, and stability. They are widely used in hydrogenation, dehydrogenation, oxidation, reduction, isomerization, aromatization, cracking, synthesis, and other reactions, playing a very important role in the fields of chemical engineering, petroleum refining, petrochemicals, pharmaceuticals, environmental protection, and new energy.

Precious metal catalysts are usually composed of precious metal active components, additives, and supports. Common methods for loading precious metals include sol fixation and impregnation. However, both methods have some shortcomings. The sol fixation method is relatively complex, involving many steps and chemical reactions, and requires strict control of temperature, pH value, reaction time, etc. In the process of sol fixation, organic macromolecules are often used as protective agents for metal particles. Due to the lack of strong interaction between the metal and the support, it is easy to cause the loss of active components during the reaction process, and residual protective agents will affect the stability and purity of the catalyst. The impregnation method is simple and easy to implement, efficient and convenient, but during the impregnation process, it is affected by solvation effects and cluster effects of active components, which makes it difficult to highly disperse the active components, resulting in a large amount of active components and low metal utilization. After immersion, high-temperature reduction of the metal is also required, which will lead to the aggregation of the loaded metal. The reduction process usually uses hydrogenation reduction, which carries certain risks.

As stated in patent CN109261145A, after obtaining the activated carbon catalyst loaded with precious metals, it needs to be reduced for 10 hours under a hydrogen atmosphere at 160° C. In patent CN104399537A, a Pd/Al₂O₃ catalyst was obtained, which needs to be reduced under a hydrogen atmosphere at 200° C. for 3 to 50 hours.

Currently, finding a simple, safe, and energy-saving technology to prepare excellent precious metal catalysts is still a research direction for catalysts. Therefore, it is of great significance to prepare precious metal catalysts without the addition of reducing agents, which can avoid dangerous hydrogenation reduction processes, reduce the agglomeration of loaded metals, and improve the quality of catalysts.

### SUMMARY OF THE INVENTION

The purpose of the present invention is to provide a method and device for treating alumina precursor using a carbon containing mixed gas, and preparing alumina support with reducing function and catalyst.

The method for preparing an alumina support with reducing function and catalyst using a carbon containing mixed gas provided by the present invention is to react the carbon containing mixed gas with an alumina precursor. The carbon containing mixed gas can adsorb on the surface of the alumina precursor, induce a change in the Al—O bond structure in the alumina support, and dissociate and activate hydroxyl groups on the surface to form an active intermediate H*. Some of the active intermediates are removed, while others migrate on the surface and eventually stabilize on the surface, forming an alumina support with reducing Al—O—H* bond structure. The alumina support is loaded with precious metal salt solution by impregnation method. During the loading process, the Al—O—H* structure on the surface of the support can reduce the precious metal on the support in situ, effectively maintaining the small size and high dispersion of the metal particles, and achieving synchronous completion of loading and reduction.

The method for preparing alumina support with reducing function and catalyst using carbon containing mixed gas is as follows:

A: Place the alumina precursor in the reactor, seal it, and first introduce the protective gas into the reactor through the deoxygenation device. Then, introduce the M gas into the reactor after deoxygenation through the deoxygenation device. Control the flow rate of M gas and protective gas through a mass flow meter to adjust the ratio of reaction gas. After the reaction gas is introduced for 8-15 minutes, use programmed heating to heat the reactor at a heating rate of 2-15° C./min, a reaction temperature of 400-600° C., a reaction time of 1-10 hours, preferably 2-6 h; a reaction gas flowrate of 10 mL/min-100 mL/min, preferably 10 mL/min-60 mL/min After the reaction is complete, cool to room temperature, quickly remove the sample and vacuum pack it for later use to obtain a reducible alumina support with a γ-type crystal structure;

The reaction gas is composed of carbon containing gas M and protective gas, wherein M accounting for 5%-60% by volume and the rest being protective gas; preferably M has a volume content of 10%-40%.

The protective gas is an inert gas, such as nitrogen or argon;

The deoxygenation device is a drying tube with 401 manganese based deoxidizer added;

The M is any one or several of CH₄, C₂H₂, CO, C₂H₄, C₂H₆. The preferred ones are any 1˜3 of CH₄, C₂H₂, CO, C₂H₄, C₂H₆.

The alumina precursor is any one of spherical boehmite, strip boehmite, powdery pseudo boehmite, and powdery aluminum hydroxide; preferably powdered aluminum hydroxide or powdered pseudo boehmite;

In the aforementioned reaction process, the carbon-containing gas molecules in the carbon—containing mixed gas adsorb onto the surface of the alumina precursor, inducing a change in its Al—O bonding structure and activating its surface hydroxyl groups, producing active intermediate H*. Some of the active H* is removed, while others migrate on the surface and re-stabilize, forming a reducing Al—O—H* bond structure.

B: Add an equal volume of precious metal impregnation solution to the alumina support in step A, where the concentration of the precious metal solution is determined according to the required content of precious metal in the final product;

For the formed alumina support, mix it evenly with a precious metal solution and place it in an oven to dry at a temperature of 20-90° C. for 6-36 hours to obtain an alumina support loaded with precious metal elements.

For powdered alumina support, a mixture of powdered alumina support and precious metal solutions is placed in a water bath, stirred with a magnetic stirrer at a speed of 100-1000 revolutions per minute, and when the solution is nearly dry, it is placed in an oven for drying to obtain alumina support loaded with precious metal elements.

During the impregnation process, metal cations gradually approach the surface of the support and migrate to the vicinity of the Al—O—H* bond due to charge interactions. The Al—O—H* structure gives electrons to the metal cations, thereby reducing them to form metal atoms and obtaining the reduced precious metal catalyst, achieving synchronous completion of loading and reduction.

The device used for preparing alumina support with reducing function using carbon containing mixed gas mentioned above, the device consists of M gas storage tank, protective gas storage tank, deoxygenation device, reactor, tail gas collection bottle and other devices. The M gas storage tank and the protective gas storage tank are respectively connected to the deoxygenation device through mass flow meters. The deoxygenation device is connected to the reactor through a gas circuit, and pressure gauges are installed at the inlet and outlet of the reactor. The bottom of the reactor is connected to the exhaust gas collector container. There are n number M gas storage tanks, depending on the number of selected M gas types, n=1-5.

The characterization of the prepared catalyst yielded the following results:

From the XRD pattern in FIG. 2, it shows that the γ-alumina support was successfully prepared by this method.

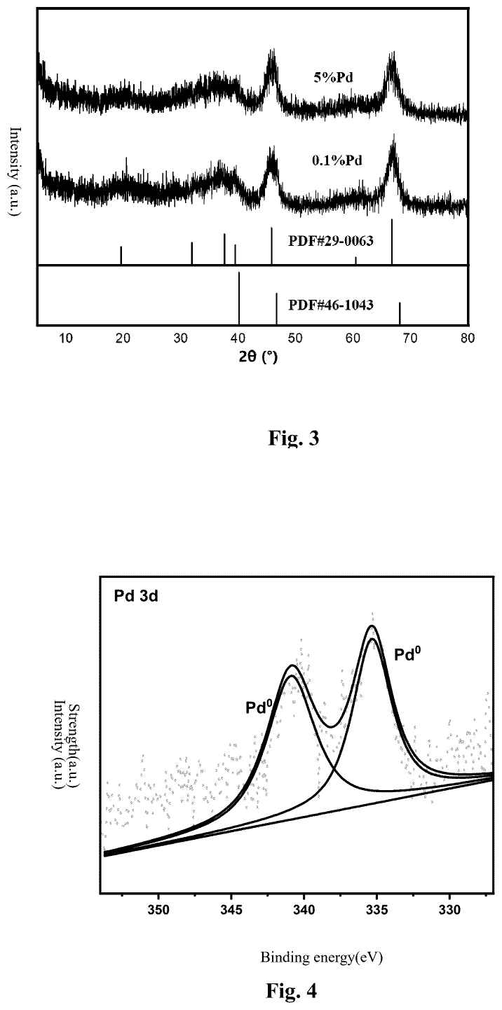

From the XRD pattern in FIG. 3, no obvious Pd particle peaks were observed, indicating that Pd is relatively evenly dispersed.

From the XPS spectra in FIGS. 4, 5, and 6, it can be seen that Ru, Pd, and Au have been reduced.

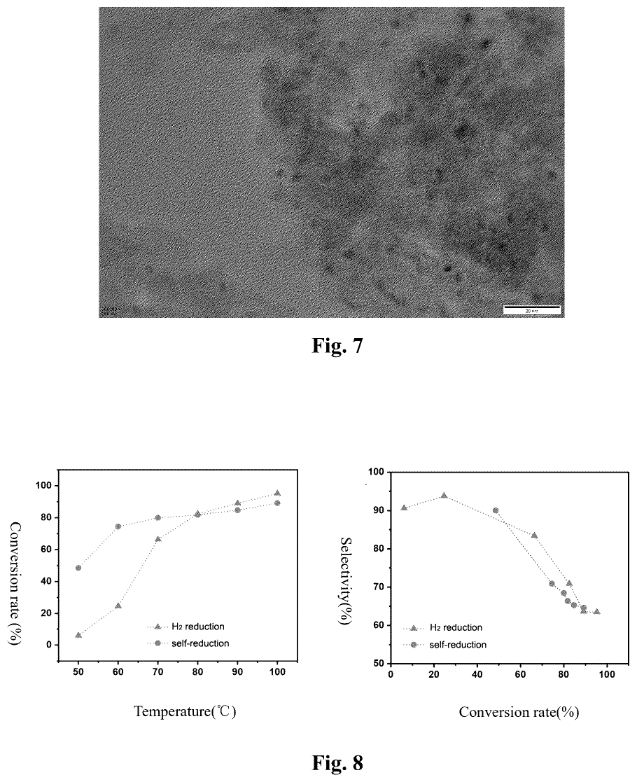

The HRTEM in FIG. 7 indicates that Pd is well dispersed and relatively uniform.

From the performance comparison in FIG. 8, it can be seen that the Pd/Al₂O₃ catalyst prepared by this method has a certain improvement in the semi hydrogenation performance of acetylene.

### Beneficial Effects:

The feature of the present invention is to use a carbon containing mixed gas to perform special surface treatment on alumina precursor during its preparation process, resulting in an alumina support with special reduction function. The alumina prepared by this method can directly reduce precious metals during the impregnation process without adding any reducing agents. Therefore, the traditional operation process of hydrogenation reduction after loading is avoided, and the agglomeration of precious metals caused by high-temperature reduction process is avoided, resulting in good dispersion of precious metals on the surface. At the same time, the high energy consumption of high-temperature reduction process and the danger of hydrogenation reduction are avoided.

The device provided for preparing alumina support with reducing function can control the proportion of reaction gas by adjusting the flow meter. The gas enters the reactor through the gas path and comes into full contact with the sample. The reaction gas is finally collected by the gas collection device and can be reused.

The catalytic performance of the catalyst prepared by the present invention was tested for acetylene semi-hydrogenation reaction. Compared with the catalyst reduced by traditional methods, the catalyst prepared by the present invention shows a certain improvement in catalytic performance.

### ILLUSTRATION DESCRIPTION

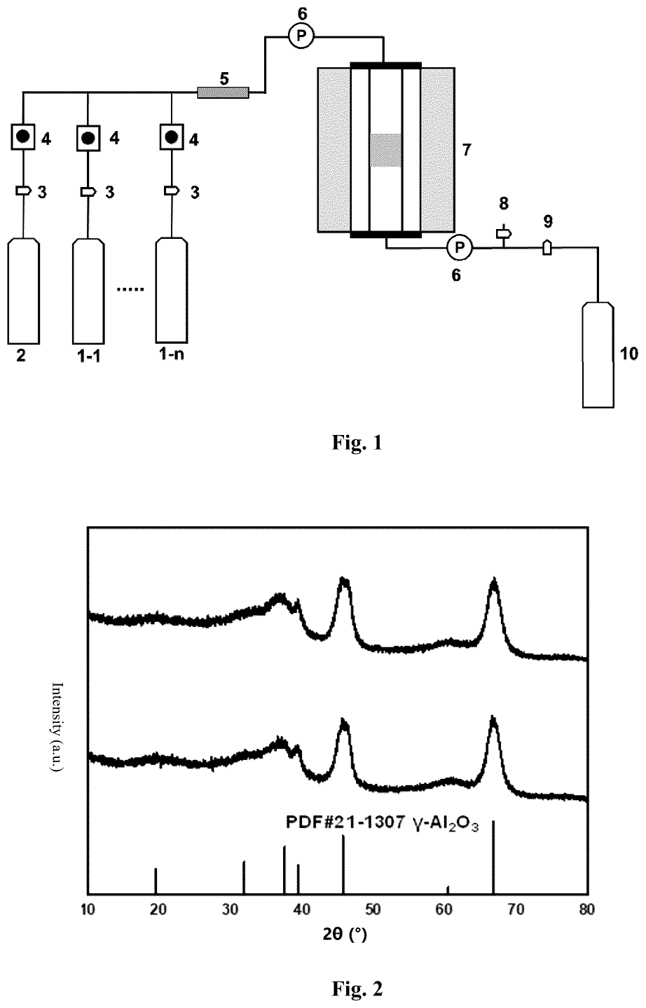

FIG. 1 shows an apparatus for preparing alumina support with reducing function. Among them, 1 is the M gas storage tank (1-1˜1-n represents the number of storage tanks), 2 is the protective gas storage tank, 3 is the inlet valve, 4 is the mass flow meter, 5 is the deoxygenation device, 6 is the pressure gauge, 7 is the reactor, 8 is the gas collection port, 9 is the outlet valve, and 10 is the exhaust gas collection bottle.

FIG. 2 shows the XRD spectra of alumina supports prepared in Examples 3 and 4.

FIG. 3 shows the XRD spectra of alumina loaded with Pd in Examples 8 and 9.

FIG. 4 shows the Pd3d XPS spectrum of alumina loaded with Pd in Example 1.

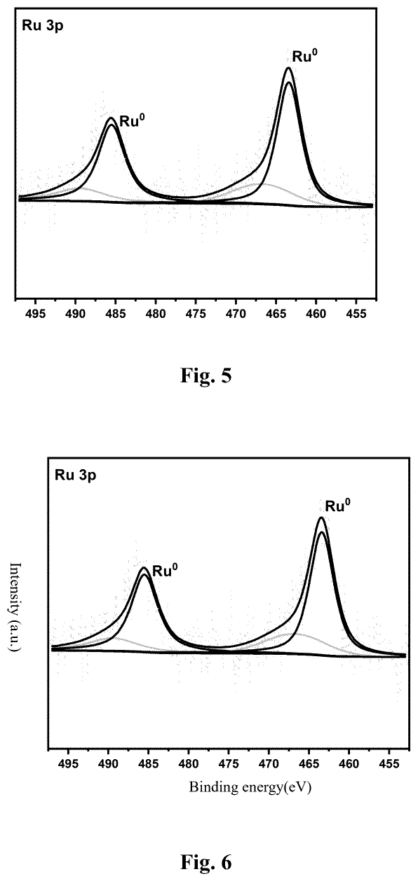

FIG. 5 shows the Ru3p XPS spectrum of alumina loaded with Ru in Example 3.

FIG. 6 shows the Au4f XPS spectrum of alumina loaded with Au in Example 4.

FIG. 7 shows the HRTEM image of alumina loaded with Pd in Example 7.

FIG. 8 shows the comparison of acetylene semi-hydrogenation catalytic performance between Pd/Al₂O₃ catalyst prepared in Example 8 and Pd/Al₂O₃ catalyst prepared by traditional hydrogenation reduction.

### DESCRIPTION OF EMBODIMENTS

In the following examples, the gas percentage content is expressed as a percentage of volume.

### Example 1

Weigh 10 g of powdered boehmite sample and place it in the reactor, seal it, and introduce the protective gas into the reactor through the deoxygenation device to check the airtightness of the device. M gas selects methane, ethane, and carbon monoxide, and passes them into the reactor through a deoxygenation device. The flow rate ratio of methane, ethane, carbon monoxide, and protective gas is controlled by a mass flow meter to be 3:7:3:7, so that the gas ratio flowing through the reactor is 15% CH₄/35% C₂H₆/15% CO/35% N₂(protective gas). Using programmed heating, raise the temperature at a rate of 5° C./min to 450° C., adjust the gas flow rate to 35 mL/min, and the reaction time to 4 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the alumina support from step A in a 100 mL beaker, add an equal volume of 0.1% sodium chloropalladate solution, and place it in a 40° C. water bath with a magnetic rotation speed of 550 r/min. After the moisture in the beaker evaporates, place the sample in a 70° C. oven to dry, obtaining alumina loaded with Pd.

### Example 2

A. Weigh 5 g of spherical boehmite sample and place it in the reactor, seal it, and introduce the protective gas into the reactor through the deoxygenation device to check the airtightness of the device. M gas selects acetylene and methane, passes them through a deoxygenation device and enters the reactor. The flow rate ratio of acetylene, methane, and protective gas is controlled by a mass flow meter to be 3:2:5, so that the gas ratio flowing through the reactor is 30% C₂H₂/20% CH₄/50% N₂(protective gas). Using programmed heating, raise the temperature at a rate of 10° C./min to 550° C., adjust the gas flow rate to 35 mL/min, and the reaction time to 3 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample in step A into a 50 mL sample tube, add an equal volume of 4% chloroauric acid solution, sonicate for 5 minutes, and immerse the sample in an 80° C. oven for 16 hours to obtain Au loaded alumina.

### Example 3

A. Weigh 10 g of powdered pseudo boehmite sample and place it in the reactor, seal it, and introduce the protective gas into the reactor through the deoxygenation device to check the airtightness of the device. M gas selects methane and acetylene, passes them through a deoxygenation device, and enters the reactor.

The flow rate ratio of methane, acetylene, and protective gas is controlled by a mass flow meter to be 7:4:9, so that the gas ratio flowing through the reactor is 35% CH₄/20% C_{Z}HZ/45% N₂(protective gas). Using programmed heating, raise the temperature at a rate of 10° C./min to 450° C., adjust the gas flow rate to 35 mL/min, and the reaction time to 3 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample from step A in a 100 mL beaker, add an equal volume of 5% ruthenium chloride solution, and place it in a 40° C. water bath with a magnetic rotation speed of 500 r/min. After the moisture in the beaker has evaporated, place the sample in an 80° C. oven to dry, obtaining Ru loaded alumina.

### Example 4

A. Weigh 4 g of powdered pseudo boehmite sample and place it in a reactor, seal it, and introduce protective gas into the reactor through a deoxygenation device to check the airtightness of the device. M gas selects methane and carbon monoxide, passes them through a deoxygenation device, and enters the reactor. The flow rate ratio of methane, carbon monoxide, and protective gas is controlled by a mass flow meter to be 11:1:8, so that the gas ratio flowing through the reactor is 55% CH₄/5% CO/40% N₂(protective gas). Using programmed heating, raise the temperature at a rate of 10° C./min to 250° C., adjust the gas flow rate to 35 mL/min, and the reaction time to 3 h. Afterwards, change the concentration of the reaction gas to 20% CH₄/80% N₂(protective gas); The gas flow rate is 50 mL/min; Heat up to 550° C. at a rate of 10 C/min for 2 hours. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample from step A in a 100 mL beaker, add an equal volume of 2% chloroauric acid solution, and place it in a 60° C. water bath with a magnetic rotation speed of 600 r/min. After the moisture in the beaker evaporates, place the sample in a 60° C. oven for drying to obtain alumina loaded with Au.

### Example 5

A. Weigh 8 g of bar shaped boehmite sample and place it in the reactor, seal it, and introduce the protective gas into the reactor through the deoxygenation device to check the airtightness of the device. M gas selects methane and passes it through a deoxygenation device before entering the reactor. The flow rate ratio of methane to protective gas is controlled by a mass flow meter to be 1:9, so that the gas ratio flowing through the reactor is 10% CH₄/90% N₂(protective gas). Using programmed heating, raise the temperature at a rate of 10° C./min to 150° C., adjust the gas flow rate to 35 mL/min, and react for 2 hours. Afterwards, change the concentration of the reaction gas to 20% CH₄/80% N₂(protective gas); The gas flow rate is 50 mL/min; Heat up to 550° C. at a rate of 10° C./min for 2 hours. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample in step A into a 50 mL sample tube, add an equal volume of 5% sodium chloropalladate solution, sonicate for 5 minutes, and immerse the sample in a 70° C. oven for 16 hours to obtain Pd loaded alumina.

### Example 6

A. Weigh 6 g of powdered aluminum hydroxide sample and place it in the reactor, seal it, and introduce the protective gas into the reactor through the deoxygenation device to check the airtightness of the device. M gas selects methane and ethane, passes them through a deoxygenation device, and enters the reactor. The flow rate ratio of methane, ethane, and protective gas is controlled by a mass flow meter to be 1:1:3, so that the gas ratio flowing through the reactor is 20% CH₄/20% C₂H₆/60% N₂(protective gas). Using programmed heating, raise the temperature at a rate of 10° C./min to 500° C., adjust the gas flow rate to 35 mL/min, and the reaction time to 3 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample from step A in a 100 mL beaker, add an equal volume of 2% sodium chloropalladate solution, and place it in a 50° C. water bath with a magnetic rotation speed of 400 r/min. After the water in the beaker evaporates, place the sample in a 70° C. oven to obtain Pd loaded alumina.

### Example 7

A. Weigh 2 g of spherical boehmite sample and place it in the reactor, seal it, and introduce the protective gas into the reactor through the deoxygenation device to check the airtightness of the device. M gas selects methane and carbon monoxide, passes them through a deoxygenation device, and enters the reactor. The flow rate ratio of methane, carbon monoxide, and protective gas is controlled by a mass flow meter to be 1:3:6, so that the gas ratio flowing through the reactor is 10% CH₄/30% CO/60% N₂(protective gas). Using programmed heating, raise the temperature at a rate of 10° C./min to 550° C., adjust the gas flow rate to 15 mL/min, and the reaction time to 4 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample in step A into a 50 mL sample tube, add an equal volume of 5% sodium chloropalladate solution, sonicate for 5 minutes, and immerse the sample in an 80° C. oven for 24 hours to obtain Pd loaded alumina.

### Example 8

A. Weigh 10 g of spherical boehmite sample and place it in the reactor, seal it, and introduce the protective gas into the reactor through the deoxygenation device to check the airtightness of the device. M gas selects ethane and acetylene, passes them through a deoxygenation device, and enters the reactor. The flow rate ratio of ethane, acetylene, and protective gas is controlled by a mass flow meter to be 4:1:5, so that the gas ratio flowing through the reactor is 40% C₂H₆/10% C₂H₂/50% N₂(protective gas). Using programmed heating, raise the temperature at a rate of 10° C./min to 500° C., adjust the gas flow rate to 25 mL/min, and the reaction time to 3 h. After the reaction is complete, cool to room temperature and quickly remove the sample to obtain a reducible alumina support, which is vacuum packed for later use. The deoxygenation device uses 401 manganese based deoxidizer for deoxygenation.

B. Place the sample in step A into a 50 mL sample tube, add an equal volume of 0.1% sodium chloropalladate solution, sonicate for 5 minutes, and immerse the sample in an 80° C. oven for 16 hours to obtain Pd loaded alumina.

### COMPARATIVE EXAMPLE

The comparative sample was prepared according to the parameters in Example 1, except that the carbon containing gas mixture was not treated.

A. Weigh 10 g of powdered boehmite sample and place it in a reactor. The protective gas is introduced into the reactor through a deoxygenation device and heated using programmed heating at a rate of 5° C./min to 450° C. Adjust the gas flow rate to 35 mL/min and the reaction time to 4 h. Obtain alumina support.

B. Place the alumina support from step A in a 100 mL beaker, add an equal volume of 0.1% sodium chloropalladate solution, and place it in a 40° C. water bath with a magnetic rotation speed of 550 r/min. After the moisture in the beaker is almost dry, place the sample in a 70° C. oven to obtain alumina loaded with Pd.

C. Place the Pd loaded alumina obtained in step B in a tube furnace, set the reaction temperature to 250° C., and calcine for 3 hours in a 10% H₂/N₂ atmosphere to obtain Pd/Al₂O₃ catalyst.

### Application Example

The catalysts prepared in Example 1 and Comparative Example 1 were respectively used in the acetylene semi-hydrogenation reaction experiment:

Weigh 0.2 g of catalyst and mix it thoroughly with 1.8 g of quartz sand with a particle size of 40-70 mesh. Load the catalyst mixture into a quartz reaction tube with a diameter of 10 mm. The catalytic performance test temperature is 50-150° C., and the gas composition in the reaction feed gas is 0.71% C₂H₂/2.86% H₂/70.72% C₂H₄/25.71% N₂(equilibrium gas). The test pressure is 1 bar and the airspeed is 3800 h⁻¹. The composition and content of reactants and products are analyzed by gas chromatography, and the data processing method is normalization. To ensure the accuracy of the test, record the results after reaching the specified temperature and holding it for 30 minutes. The test is conducted in 3 groups, and the average value is the catalytic performance data at that catalytic temperature. The results are shown in FIG. 8.

From FIG. 8, it can be seen that compared to the Pd/Al₂O₃ catalyst prepared by traditional methods, the Pd/Al₂O₃ catalyst prepared by impregnation in-situ self reduction method has better hydrogenation activity in the low-temperature region.

1. A method for preparing alumina support with reducing function and catalyst using a carbon containing mixed gas, characterized by the following steps:
A: Place the alumina precursor in the reactor, seal it, and first introduce the protective gas into the reactor through the deoxygenation device. Then, introduce the M gas into the reactor after deoxygenation through the deoxygenation device. Control the flow rate of M gas and protective gas through a mass flow meter to adjust the ratio of reaction gas. After the reaction gas is introduced for 8-15 minutes, use programmed heating to heat the reactor at a heating rate of 2-15° C./min, a reaction temperature of 400-60° C., a reaction time of 1-10 hours, and a reaction gas flow rate of 10 mL/min-100 mL/min; After the reaction is complete, cool to room temperature, quickly remove the sample and vacuum pack it for later use to obtain a reducible alumina carrier with a γ-type crystal structure;
The reaction gas is composed of carbon containing gas M and protective gas, wherein M accounting for 5%-60% by volume and the rest being protective gas;
The protective gas is an inert gas, such as nitrogen or argon;
The deoxygenation device is a drying tube with 401 manganese based deoxidizer added;
The M is any one or several of CH₄, C₂H₂, CO, C₂H₄, C₂H₆;
The alumina precursor is any one of spherical boehmite, strip boehmite, powdery pseudo boehmite, and powdery aluminum hydroxide; preferably powdered aluminum hydroxide or powdered pseudo boehmite;
In the aforementioned reaction process, the carbon-containing gas molecules in the carbon-containing mixed gas adsorb onto the surface of the alumina precursor, inducing a change in its Al—O bonding structure and activating its surface hydroxyl groups, producing active intermediate H*. Some of the active H* is removed, while others migrate on the surface and re-stabilize, forming a reducing Al—O—H* bond structure;
B: Add an equal volume of precious metal impregnation solution to the alumina support in step A, where the concentration of the precious metal solution is determined according to the required content of precious metal in the final product;
For the formed alumina support, mix it evenly with a precious metal solution and place it in an oven to dry at a temperature of 20-90° C. for 6-36 hours to obtain an alumina support loaded with precious metal elements;
For powdered alumina support, a mixture of powdered alumina support and precious metal solutions is placed in a water bath, stirred with a magnetic stirrer at a speed of 100-1000 revolutions per minute, and when the solution is nearly dry, it is placed in an oven for drying to obtain alumina carrier loaded with precious metal elements;
During the impregnation process, metal cations gradually approach the surface of the support and migrate to the vicinity of the Al—O—H* bond due to charge interactions. The Al—O—H* structure gives electrons to the metal cations, thereby reducing them to form metal atoms and obtaining the reduced precious metal catalyst, achieving synchronous completion of loading and reduction.

2. The method for preparing alumina support with reducing function and catalyst using a carbon containing mixed gas according to claim 1, characterized in that: the programmed heating process in step A has a reaction time of 2-6 hours; The reaction gas flow rate is 10 mL/min-60 mL/min;
The volume content of M in the reaction gas is 10%-40%, and the rest is protective gas;
The M is any one, two or three of CH₄, C₂H₂, CO, C₂H₄, C₂H₆.

3. A device for preparing an alumina support with reducing function using a carbon containing mixed gas according to claim 1, comprising an M gas storage tank, a protective gas storage tank, a deoxygenation device, a reactor, a tail gas collection bottle, and other devices; The M gas storage tank and the protective gas storage tank are respectively connected to the deoxygenation device through mass flow meters. The deoxygenation device is connected to the reactor through a gas circuit, and pressure gauges are installed at the inlet and outlet of the reactor. The bottom of the reactor is connected to the exhaust gas collector container;
There are n number M gas storage tanks, depending on the number of selected M gas types, n=1-5.
